(19) United States
(12) Patent Application Publication
(10) Pub. No.: US 2025/0382286 A1
(43) Pub. Date: Dec. 18, 2025

(54) CO-CRYSTAL OF AFICAMTEN, AND PREPARATION METHOD THEREFOR AND USE THEREOF

(71) Applicant: CRYSTAL PHARMACEUTICAL (SUZHOU) CO., LTD., Suzhou (CN)

(72) Inventors: Yuxing ZHANG, Suzhou (CN); Jing ZHANG, Suzhou (CN); Liping MENG, Suzhou (CN)

(73) Assignee: CRYSTAL PHARMACEUTICAL (SUZHOU) CO., LTD., Suzhou (CN)

(21) Appl. No.: 19/315,816

(22) Filed: Sep. 1, 2025

Related U.S. Application Data
(63) Continuation of application No. PCT/CN2024/078653, filed on Feb. 27, 2024.

(30) Foreign Application Priority Data
Mar. 2, 2023 (CN) 202310191283.1

(51) Int. Cl. C07D 413/12; A61K 31/4245; C07C 59/225

(52) U.S. Cl. CPC C07D 413/12; A61K 31/4245; C07C 59/225; C07B 2200/13

(57) ABSTRACT

The present disclosure relates to co-crystal of Aficamten and tartaric acid, and preparation methods thereof, pharmaceutical compositions containing the crystalline forms, and uses of the co-crystal for preparing cardiac myosin inhibitor and drugs for treating hypertrophic cardiomyopathy.

### CROSS-REFERENCE TO RELATED APPLICATIONS

This application is a Continuation of International Application No. PCT/CN2024/078653, filed on Feb. 27, 2024, which claims priority to Chinese patent application No. 202310191283.1, filed on Mar. 2, 2023, the contents of each of which are incorporated herein by reference in their entirety.

### TECHNICAL FIELD

The present disclosure pertains to the field of chemical crystallography, particularly relates to co-crystal of Aficamten, preparation method and use thereof.

### BACKGROUND

The cardiac sarcomere is composed of a reticular network of contractile and structural proteins that regulate myocardial function. Abnormalities in the cardiac sarcomere have been identified as the cause of various heart diseases, such as hypertrophic cardiomyopathy (HCM). HCM is a disease characterized by abnormal thickening or enlargement of the heart muscle, primarily resulting from sarcomere dysfunction. This thickening causes the inside of the left ventricle of the heart to become smaller and stiffer, preventing it from relaxing and filling with blood and limiting the heart's pumping function and resulting in symptoms such as chest pain, dizziness, shortness of breath, or fainting. Current sarcomere-targeting drugs lack sufficient selectivity for cardiac tissue, which can lead to side effects and limit their use. Given the limitations of existing drugs, there remains a need for new therapies to treat cardiac diseases.

Aficamten is a novel, oral small-molecule cardiac myosin inhibitor developed by Cytokinetics for the treatment of HCM. It has demonstrated positive outcomes in Phase 3 clinical studies. The chemical name of Aficamten is (R)-N-(5-(5-ethyl-1,2,4-oxadiazol-3-yl)-2,3-dihydro-1H-inden-1-yl)-1-methyl-1H-pyrazole-4-carboxamide (Referred to as Compound I), and the structure is shown as the follows:

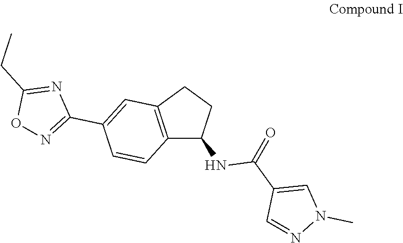 [SMILES: CCc1nc(-c2ccc3c(c2)CC[C@H]3NC(=O)c2cnn(C)c2)no1]

It is well known in the field that drug polymorphism is a common phenomenon in small molecule drug development, and it is an important factor affecting drug quality. A crystalline form is a solid material whose constituents are arranged in a highly ordered microscopic structure, forming a crystal lattice that extends in all directions. Polymorphism refers to the phenomenon that a compound exists in more than one crystalline form. Compounds may exist in one or more crystalline forms, but their existence and characteristics cannot be predicted with any certainty.

Different crystalline forms of an active pharmaceutical ingredient (API) have different physicochemical properties, such as chemical stability and solubility, which can affect drug's in vivo dissolution and absorption and will further affect drug's clinical efficacy and safety to some extent. In addition, different solid forms of APIs may have different manufacturability characteristics, including yield, purification property, filtration property, drying property, milling behavior, and stability under compression pressure during tableting, which can impact the processing and handling during drug production. Therefore, different solid forms of APIs may possess unique properties, offering opportunities to improve the performance of pharmaceutical products.

The prior art WO2021011807A1 disclosed Forms I-VI of Compound I, and Form IV is the most stable form. However, the inventors of the present disclosure have found that Form IV exhibits low solubility. To find a novel solid form that can improve drug performance, the inventors of the present disclosure surprisingly obtained the co-crystal of Compound I and tartaric acid in the present disclosure. According to FDA Regulatory Classification of Pharmaceutical Co-Crystals Guidance for Industry, pharmaceutical co-crystals are crystalline materials composed of two or more different molecules (one of which is the API) in a defined stoichiometric ratio within the same crystal lattice that are associated by nonionic and noncovalent bonds. Pharmaceutical co-crystals can be tailored to enhance drug product bioavailability and stability and to enhance the processability of APIs during drug product manufacture. Another advantage of co-crystals is that they generate better solid-state forms for APIs that lack ionizable functional groups, which is a prerequisite for salt formation. The inventors of the present disclosure surprisingly obtained co-crystal of Compound I and co-crystal in the present disclosure, which have advantages in at least one aspect of solubility, hygroscopicity, purification ability, stability, adhesiveness, compressibility, flowability, in vitro and in vivo dissolution, and bioavailability, etc. In particular, the co-crystal of the present disclosure has advantages such as high solubility, good flowability, low hygroscopicity and good stability, which solves the problem existing in prior arts and is of great significance for the development of drugs containing Compound I.

### SUMMARY

The present disclosure is to provide co-crystal of Compound I, preparation method and pharmaceutical compositions comprising the co-crystal.

According to the objective of the present disclosure, co-crystal of Compound I and tartaric acid is provided by the present disclosure.

According to the objective of the present disclosure, co-crystal Form CSI of Compound I and tartaric acid is provided by the present disclosure (hereinafter referred to as Form CSI).

In one aspect provided herein, the X-ray powder diffraction pattern of Form CSI comprises characteristic peaks at 2theta values of 12.2°±0.2°, 14.7°±0.2° and 19.1°±0.2° using CuKα radiation.

Furthermore, the X-ray powder diffraction pattern of Form CSI comprises one or two or three characteristic peaks at 2theta values of 7.3°±0.2°, 8.9°±0.2° and 15.6°±0.2° using CuKα radiation. Preferably, the X-ray powder diffraction pattern of Form CSI comprises characteristic peaks at 2theta values of 7.3°±0.2°, 8.9°±0.2° and 15.6°±0.2° using CuKα radiation.

Furthermore, the X-ray powder diffraction pattern of Form CSI comprises one or two or three characteristic peaks at 2theta values of 10.9°±0.2°, 12.6°±0.2° and 22.8°±0.2° using CuKα radiation. Preferably, the X-ray powder diffraction pattern of Form CSI comprises characteristic peaks at 2theta values of 10.9°±0.2°, 12.6°±0.2° and 22.8°±0.2° using CuKα radiation.

In another aspect provided herein, the X-ray powder diffraction pattern of Form CSI comprises one or two or three or four or five or six or seven or eight or nine characteristic peaks at 2theta values of 12.2°±0.2°, 14.7°±0.2°, 19.1°±0.2°, 7.3°±0.2°, 8.9°±0.2°, 15.6°±0.2°, 10.9°±0.2°, 12.6°±0.2°, 22.8°±0.2°, 10.2°±0.2°, 18.0°±0.2° using CuKα radiation.

Without any limitation being implied, Form CSI is an anhydrous co-crystal of Compound I and tartaric acid.

Without any limitation being implied, Form CSI is preferably a co-crystal of Compound I with L-tartaric acid, DL-tartaric acid or D-tartaric acid, more preferably with L-tartaric acid.

Without any limitation being implied, the molar ratio of tartaric acid to Compound I in Form CSI is preferably from 0.4 to 0.6, and more preferably 0.5.

Without any limitation being implied, an XRPD pattern of Form CSI is substantially as depicted in FIG. 1 using CuKα radiation.

Without any limitation being implied, a TGA curve of Form CSI is substantially as depicted in FIG. 2, which shows almost no weight loss when heated to about 100° C.

Without any limitation being implied, a DSC curve of Form CSI is substantially as depicted in FIG. 3, which shows an endothermic peak with an onset temperature of about 163° C. and a peak temperature at about 166° C.

According to the objective of the present disclosure, a process for preparing Form CSI is also provided. The process comprises: Stirring Compound I and tartaric acid in a ketone to obtain Form CSI.

Furthermore, said ketone is preferably methyl isobutyl ketone. Said tartaric acid is preferably L-tartaric acid, DL-tartaric acid or D-tartaric acid, more preferably L-tartaric acid.

According to the objective of the present disclosure, the present disclosure provides a pharmaceutical composition comprising a therapeutically effective amount of co-crystal of Compound I and tartaric acid, and pharmaceutically acceptable excipients.

Furthermore, the present disclosure provides a pharmaceutical composition comprising a therapeutically effective amount of Form CSI and pharmaceutically acceptable excipients.

According to the objective of the present disclosure, the present disclosure provides a method of preparing cardiac myosin inhibitor drugs comprising co-crystal of Compound I and tartaric acid.

Furthermore, the present disclosure provides a method of preparing cardiac myosin inhibitor drugs comprising Form CSI.

According to the objective of the present disclosure, the present disclosure provides a method of preparing drugs for treating hypertrophic cardiomyopathy comprising co-crystal of Compound I and tartaric acid.

Furthermore, the present disclosure provides a method of preparing drugs for treating hypertrophic cardiomyopathy comprising Form CSI.

Form CSI of the present disclosure exhibits the following unexpected technical effects:

Form CSI exhibits high solubility. Compared with the prior art, Form CSI shows higher solubility in FaSSGF, FaSSIF, FeSSIF and water, which is beneficial for enhancing drug absorption in the human body and improving bioavailability.

Form CSI has good flowability. Compared with the prior art, its enhanced flowability helps prevent blockage of manufacturing equipment and improves production efficiency. Moreover, it ensures the content uniformity of the drug product, reduces the weight variation of the drug product and improves product quality.

Form CSI has almost no hygroscopicity. Test results show that the weight gain of Form CSI at 80% RH is only 0.18%. Form CSI with low hygroscopicity is not demanding on the production and storage conditions, which reduces the cost of production, storage and quality control, and has strong economic value.

Form CSI has good stability.

Form CSI has good humidity stability. The crystalline form remains unchanged after undergoing a humidity cycle of 0% RH-95%RH-0%RH.

Form CSI has good physical and chemical stability. Crystalline form of Form CSI doesn't change for at least 9 months when stored under conditions of 25° C./60% RH and 40° C./75% RH. And the chemical purity remains substantially unchanged during storage. Crystalline form of Form CSI doesn't change for at least 3 months when stored under conditions of 60° C./75% RH. And the chemical purity remains substantially unchanged during storage.

Form CSI has good stability under mechanical force. The crystalline form of Form CSI doesn't change after ball milling.

High humidity conditions caused by seasonal variations, regional climate differences, and environmental factors can affect the storage, transportation, and manufacturing of APIs. In addition, grinding or milling of APIs is often required during formulation processing. Form CSI has good stability, which helps avoid the impact on drug quality due to crystal transformation during storage, transportation, and manufacturing. Besides, it reduces the risk of decreased crystalline and undesired polymorphic transitions during formulation processing. As a result, it ensures consistent and controllable quality of APIs, minimizes quality fluctuations, bioavailability changes and toxicity caused by crystal transformation.

### BRIEF DESCRIPTION OF THE DRAWINGS

FIG. 1 shows an XRPD pattern of Form CSI.

FIG. 2 shows a TGA curve of Form CSI.

FIG. 3 shows a DSC curve of Form CSI.

FIG. 4 shows a DVS plot of Form CSI.

FIG. 5 shows an XRPD overlay of Form CSI before and after DVS (from top to bottom: before DVS, after DVS).

FIG. 6 shows an XRPD overlay of Form CSI before and after ball milling (from top to bottom: before ball milling, after ball milling).

FIG. 7 shows an XRPD pattern overlay of Form CSI before and after storage under different conditions (from top to bottom: initial, 25° C./60% RH for 9 months, 40° C./75% RH for 9 months, 60° C./75% RH for 3 months).

### DETAILED DESCRIPTION

The present disclosure is further illustrated by the following examples which describe the preparation and use of the crystalline form of the present disclosure in detail. It is obvious to those skilled in the art that changes in the materials and methods can be accomplished without departing from the scope of the present disclosure.

The abbreviations used in the present disclosure are explained as follows:
XRPD: X-ray Powder Diffraction
DSC: Differential Scanning Caborimetry
TGA: Thermo Gravimetric Analysis
DVS: Dynamic Vapor Sorption
HPLC: High Performance Liquid Chromatography
¹H NMR: Proton Nuclear Magnetic Resonance
RH: Relative Humidity
Instruments and methods used for data collection:
XRPD patterns in the present disclosure were acquired by a Bruker D8 ADVANCE X-ray powder diffractometer. The parameters of the X-ray powder diffraction method are as follows:
X-Ray source: Cu, Kα
Kα1 (Å): 1.54060; Kα2 (Å): 1.54439
Kα2/Kα1 intensity ratio: 0.50
Voltage: 40 kV
Current: 40 mA
Scan range (2θ): from 4.0 degree to 40.0 degree
TGA data in the present disclosure were acquired by a TA Q500. The parameters of the TGA method of the present disclosure are as follows:
Heating rate: 10° C./min
Purge gas: N₂
DSC data in the present disclosure were acquired by a TA Q2000. The parameters of the DSC method of the present disclosure are as follows:
Heating rate: 10° C./min
Purge gas: N₂

DVS data in the present disclosure were measured via an SMS (Surface Measurement Systems Ltd.) intrinsic DVS instrument. The instrument control software is DVS-Intrinsic control software. Typical Parameters for DVS test are as follows:
Temperature: 25° C.
Gas and flow rate: N₂, 200 mL/min
RH range: 0% RH to 95% RH

¹H NMR data in the present disclosure were collected from a Bruker Avance II DMX 400M HZ NMR spectrometer. 1-5 mg of sample was weighed and dissolved with 0.5 mL of deuterated dimethyl sulfoxide to obtain a solution with a concentration of 2-10 mg/mL.

The parameters of kinetic solubility in the present disclosure are shown in Table 1.

TABLE 1

| Instrument | Agilent 1260 | |
|---|---|---|
| Column | Waters XBridge C18, 4.6 mm × 150 mm, 5.0 μm | |
| Mobile phase | A: 0.1% trifluoroacetic acid aqueous solution | |
| | B: 0.1% trifluoroacetic acid in acetonitrile | |
| | Time (min) | % B |
| Isocratic elution | 0.00 | 55 |
| | 7.00 | 55 |

| | |
|---|---|
| Run time | 7.0 min |
| Post time | 0.0 min |
| Flow rate | 1.0 mL/min |
| Injection volume | 5 μL |
| Detector wavelength | 248 nm |
| Column temperature | 40° C. |
| Sampler temperature | Room Temperature |
| Diluent | Acetonitrile/water (80:20, v/v) |

The parameters of related substance detection in the present disclosure are shown in Table 2.

TABLE 2

| Instrument | Agilent 1260 | |
|---|---|---|
| Column | Waters XBridge C18, 4.6 mm × 150 mm, 3.5 μm | |
| Ghost-Buster column | A: 0.1% trifluoroacetic acid aqueous solution | |
| | B: 0.1% trifluoroacetic acid in acetonitrile | |
| | Time (min) | % B |
| Gradient | 0.0 | 10 |
| | 20.0 | 90 |
| | 25.0 | 90 |
| | 25.1 | 10 |
| | 30.0 | 10 |

| | |
|---|---|
| Run time | 1.0 mL/min |
| Flow rate | 5 μL |
| Injection volume | 248 nm |
| Column temperature | 40° C. |
| Sample pan | Room Temperature |
| temperature | |
| Diluent | Acetonitrile/water (80:20, v/v) |

Said “stirring” is accomplished by using a conventional method in the field such as magnetic stirring or mechanical stirring and the stirring speed is 50 to 1800 r/min. Preferably the magnetic stirring speed is 300 to 900 r/min, and mechanical stirring speed is 100 to 300 r/min.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “drying” is accomplished by using a conventional method in the field such as vacuum drying, blast drying or free-air drying. The drying temperature can be room temperature or higher. Preferably the drying temperature is from room temperature to about 60° C., or to 50° C., or to 40° C. The drying time can be 2 to 48 hours, or overnight. Drying is accomplished in a fume hood, forced air convection oven or vacuum oven.

Said “co-crystal of Compound I and tartaric acid” refers to a crystalline material composed of Compound I and tartaric acid in a defined stoichiometric ratio within the same crystal lattice that are associated by nonionic and noncovalent bonds.

Said “characteristic peak” refers to a representative diffraction peak used to distinguish crystals, which usually can have a deviation of ±0.2° using CuKα radiation.

In the present disclosure, “crystal” or “crystalline form” refers to the crystal or the crystalline form being identified by the X-ray diffraction pattern shown herein. Those skilled in the art are able to understand that the X-ray powder diffraction pattern depends on the instrument conditions, the sample preparation and the purity of samples. The relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSI of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

Unless otherwise specified, the following examples were conducted at room temperature and ambient humidity. Said “room temperature” is not a specific temperature, but a temperature range of 10-30° C.

According to the present disclosure, Compound I and/or its salt used as raw materials includes, but is not limited to solid (crystalline and amorphous), oil, liquid form or solution. Preferably, Compound I used as the raw material is a solid. Raw materials of Compound I and/or a salt thereof used in the following examples were prepared by known methods in prior arts, for example, the method disclosed in WO2021011807A1.

### EXAMPLE 1 PREPARATION OF FORM CSI

203.4 mg of Compound I and 45.5 mg of L-tartaric acid were weighed into a glass vial, followed by addition of 6 mL of methyl isobutyl ketone. The mixture was stirred at room temperature for 1 day. The solid was separated and vacuum dried at 30° C. for about 2 h to obtain a dried solid.

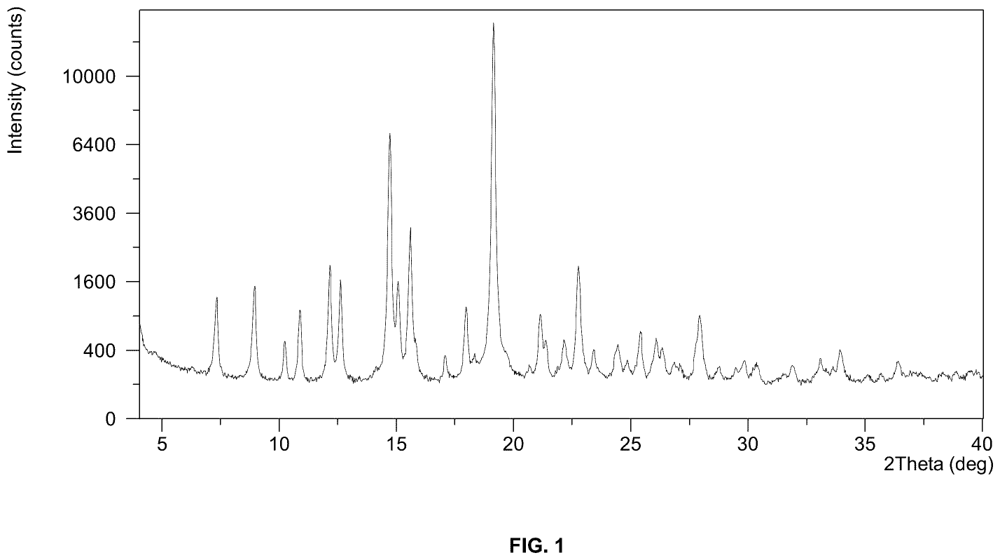

The obtained dried solid was confirmed as Form CSI. The XRPD pattern is depicted in FIG. 1, and the XRPD data are listed in Table 3.

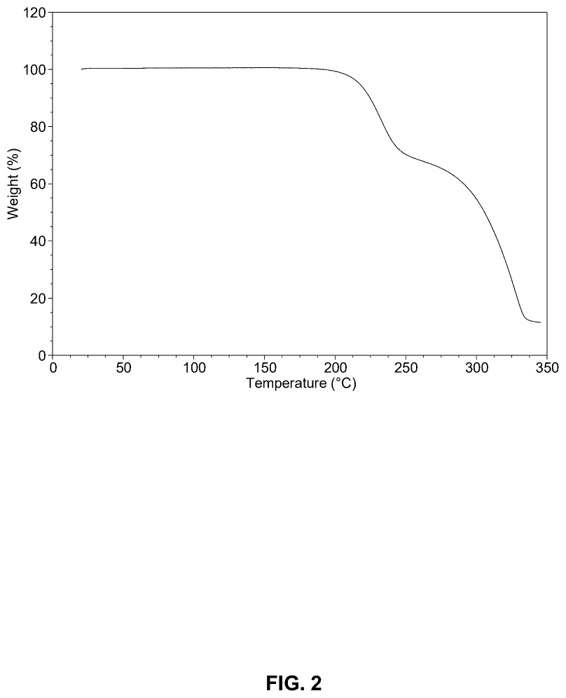

The TGA curve is depicted in FIG. 2, which shows almost no weight loss when heated to 100° C. Form CSI is an anhydrous co-crystal of Compound I and L-tartaric acid.

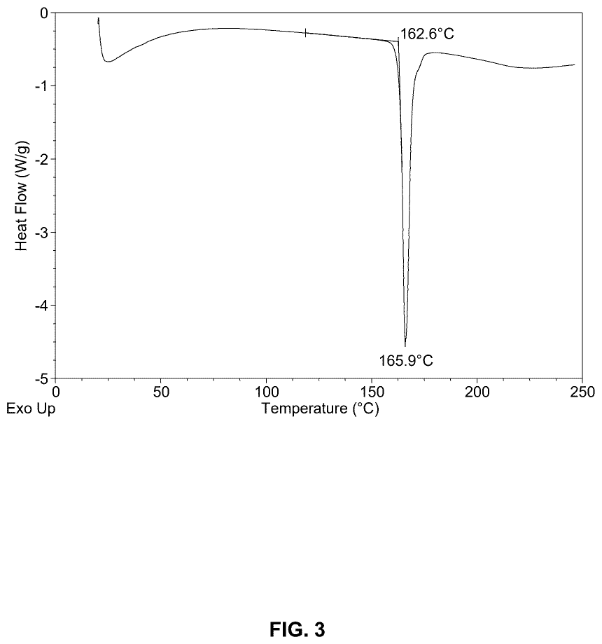

The DSC curve is depicted in FIG. 3, which shows an endothermic peak with an onset temperature of about 163° C. and a peak temperature at about 166° C.

The ¹H NMR data are: ¹H NMR (400 MHZ, DMSO-d6) δ 8.44 (d, J=8.4 Hz, 1H), 8.18 (s, 1H), 7.89 (s, 2H), 7.85 (dd, J=7.9, 1.5 Hz, 1H), 7.35 (d, J=7.8 Hz, 1H), 5.54 (q, J=8.3 Hz, 1H), 4.31 (s, 1H), 3.85 (s, 3H), 3.11-3.04 (m, 1H), 3.00 (q, J=7.5 Hz, 2H), 2.96-2.85 (m, 1H), 2.48-2.42 (m, 1H), 1.96 (dq, J=12.5, 9.0 Hz, 1H), 1.34 (t, J=7.5 Hz, 3H). The signal at 4.31 ppm is attributed to the two hydrogens on the α-carbon of the carboxyl group in L-tartaric acid, corresponding to 0.5 molar equivalents of L-tartaric acid.

TABLE 3

| | | Relative |
|---|---|---|
| 2θ (°) | d-spacing (Å) | intensity (%) |
| 7.3 | 12.1 | 8.2 |
| 8.9 | 9.9 | 10.3 |
| 10.2 | 8.6 | 2.8 |
| 10.9 | 8.1 | 6.7 |
| 12.2 | 7.3 | 14.1 |
| 12.6 | 7.0 | 11.4 |
| 14.7 | 6.0 | 51.5 |
| 15.1 | 5.9 | 11.2 |
| 15.6 | 5.7 | 22.5 |
| 17.1 | 5.2 | 1.6 |
| 18.0 | 4.9 | 7.1 |
| 19.1 | 4.6 | 100.0 |
| 20.7 | 4.3 | 0.8 |
| 21.1 | 4.2 | 6.1 |
| 21.4 | 4.2 | 3.0 |
| 22.2 | 4.0 | 2.9 |
| 22.8 | 3.9 | 14.0 |
| 23.4 | 3.8 | 2.1 |
| 24.4 | 3.6 | 2.6 |
| 24.8 | 3.6 | 1.2 |
| 25.4 | 3.5 | 3.9 |
| 26.1 | 3.4 | 3.1 |
| 26.3 | 3.4 | 2.3 |
| 26.8 | 3.3 | 1.1 |
| 27.9 | 3.2 | 6.0 |
| 28.7 | 3.1 | 0.8 |
| 29.8 | 3.0 | 1.2 |
| 30.4 | 2.9 | 1.0 |
| 31.9 | 2.8 | 0.9 |
| 33.1 | 2.7 | 1.4 |
| 33.9 | 2.6 | 2.1 |
| 36.4 | 2.5 | 1.1 |
| 38.3 | 2.4 | 0.2 |

### EXAMPLE 2 KINETIC SOLUBILITY OF FORM CSI

An adequate amount of Form CSI of the present disclosure and Form IV in the prior art was separately dispersed in 1.0 mL of FaSSGF, FaSSIF, FeSSIF and water to prepare suspensions. After equilibrating at 37° C. for 1 h, the concentration of Compound I (μg/mL) in the solution was measured by HPLC. The results are shown in Table 4. The results indicate that Form CSI exhibits high solubility in all tested media.

TABLE 4

| | Solubility of Form IV | Solubility of Form |
|---|---|---|
| Media | in the prior art (μg/mL) | CSI (μg/mL) |
| FaSSGF | 29.9 | 35.1 |
| FaSSIF | 31.9 | 42.3 |
| FeSSIF | 103.3 | 163.4 |
| Water | 33.8 | 45.1 |

### EXAMPLE 3 FLOWABILITY OF FORM CSI

Approximately 400 mg of sample was weighed into a 5-mL graduated cylinder, and the volume before tapped (Vo) was recorded. Then the sample was tapped for 1250 times by ZS-2E tap density tester and the volume after tapped (Vt) was recorded. The compressibility index (c) was calculated using the formula: c=(V₀−V_{f})/V₀*100%. A smaller compressibility index indicates better flowability. The results are shown in Table 5 and indicate that Form CSI has better flowability.

TABLE 5

| | Weight | Pre-tapping | Post-tapping | Carr |
|---|---|---|---|---|
| Solid form | (g) | volume (mL) | volume (mL) | index |
| Form IV | 0.4274 | 3.60 | 2.70 | 25.0% |
| Form CSI | 0.4031 | 3.90 | 3.05 | 21.8% |

### EXAMPLE 4 HYGROSCOPICITY AND HUMIDITY STABILITY OF FORM CSI

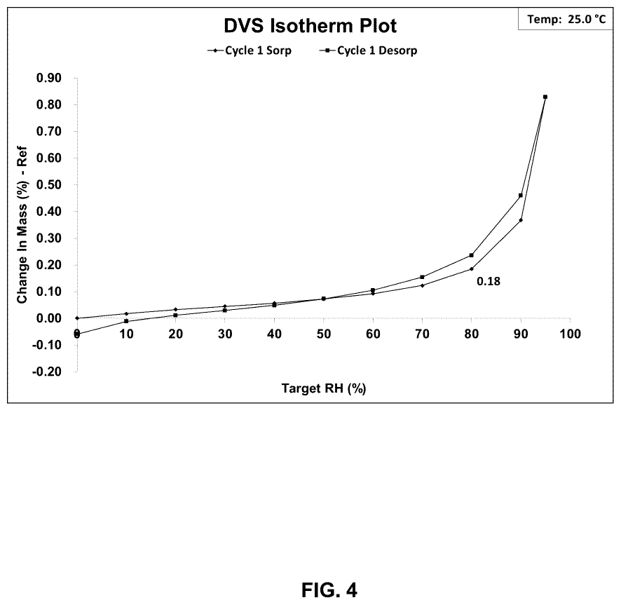

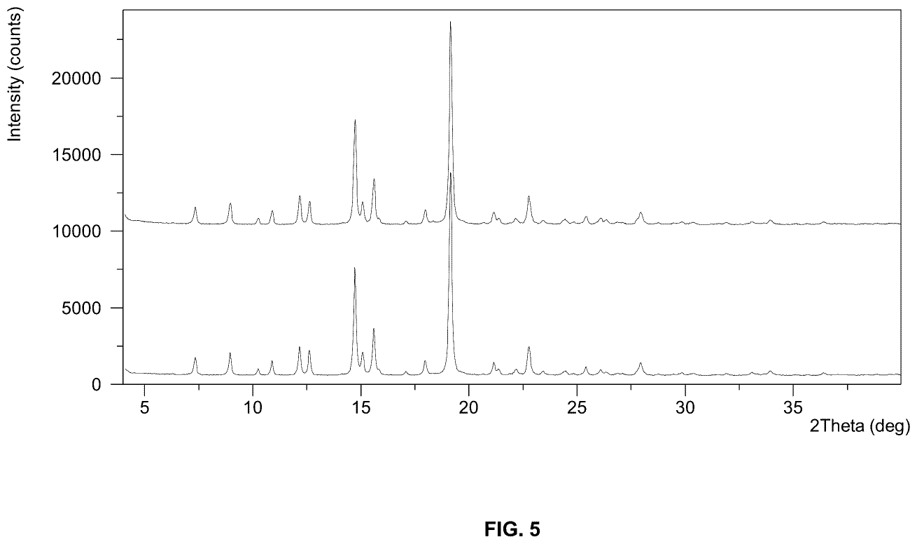

DVS analyzer was applied to evaluate the hygroscopicity of Form CSI with an appropriate amount. The mass changes at each relative humidity were recorded in a cycle of 0% RH-95%RH-0%RH. The crystalline form of the sample before and after DVS were tested by XRPD. The DVS curve of Form CSI is shown in FIG. 4, and the XRPD patterns before and after DVS are depicted in FIG. 5. The results indicate that Form CSI is almost non-hygroscopic, with a mass increase of 0.18% after reaching equilibrium at 80% RH. Additionally, Form CSI exhibits good humidity stability, as its crystalline form remains unchanged after undergoing a humidity cycle of 0% RH-95%RH-0%RH.

### EXAMPLE 5 STABILITY OF FORM CSI UNDER MECHANICAL FORCE

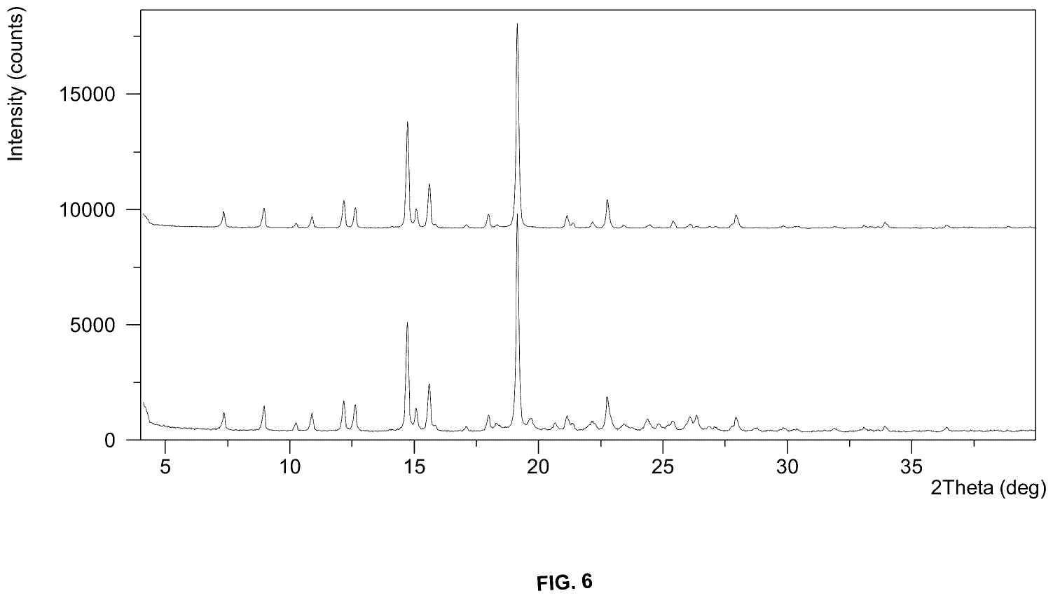

An appropriate amount of Form CSI was milled for 5 minutes at a vibration speed of 500 rpm by a ball mill. The crystalline form of the sample before and after ball milling were tested by XRPD, and the XRPD overlay is depicted in FIG. 6. The results show that no form change of Form CSI is observed after ball milling, and Form CSI has good stability under mechanical force.

### EXAMPLE 6 PHYSICAL AND CHEMICAL STABILITY OF FORM CSI

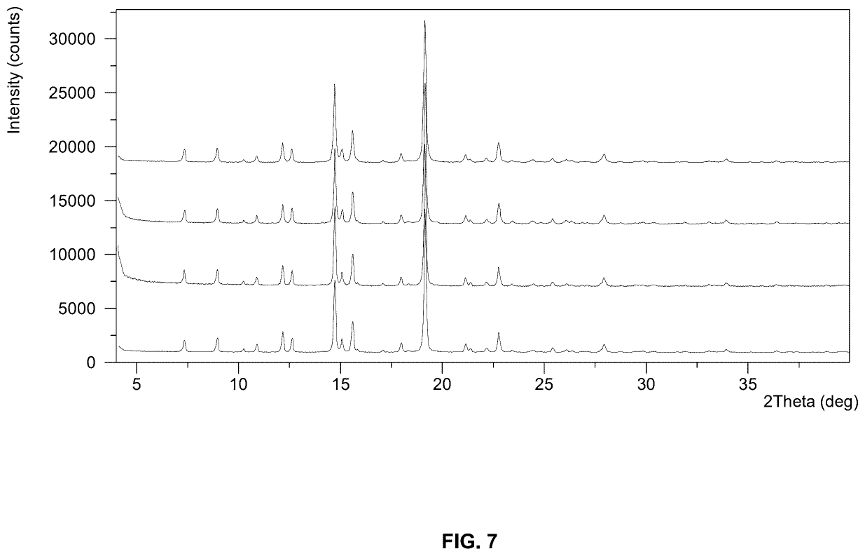

An appropriate amount of Form CSI was stored in a sealed container under different conditions of 25° C./60% RH, 40° C./75% RH and 60° C./75% RH. Chemical purity and crystalline form were tested by HPLC and XRPD. The results are shown in Table 6, and the XRPD overlay is depicted in FIG. 7.The results show that Form CSI is stable for at least 9 months at 25° C./60% RH and 40° C./75% RH, indicating Form CSI has good stability under long-term and accelerated conditions. Form CSI is stable after storage for at least 3 months at 60° C./75% RH, indicating Form CSI has good stability under stress conditions.

TABLE 6

| Initial form | Conditions | Time | Solid form | Purity |
|---|---|---|---|---|
| Form CSI | Initial | — | Form CSI | 99.98% |
| | 25° C./60% RH | 9 months | Form CSI | 99.98% |
| | 40° C./75% RH | 9 months | Form CSI | 99.99% |
| | 60° C./75% RH | 3 months | Form CSI | 99.98% |

The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1. A co-crystal of Compound I and tartaric acid, 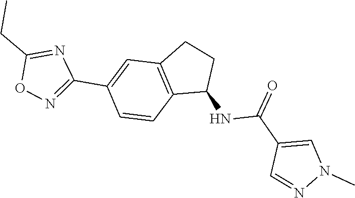 [SMILES: CCc1nc(-c2ccc3c(c2)CC[C@H]3NC(=O)c2cnn(C)c2)no1]
Compound I.

2. The co-crystal according to claim 1, wherein the X-ray powder diffraction pattern comprises characteristic peaks at 2theta values of 12.2°±0.2°, 14.7±0.2° and 19.1°±0.2° using Cu-Kα radiation.

3. The co-crystal according to claim 2, wherein the X-ray powder diffraction pattern comprises at least one characteristic peak at 2theta values of 7.3°±0.2°, 8.9±0.2° and 15.6°±0.2° using Cu-Kα radiation.

4. The co-crystal according to claim 2, wherein the X-ray powder diffraction pattern comprises at least one characteristic peak at 2theta values of 10.9°±0.2°, 12.6°±0.2° and 22.8°±0.2° using Cu-Kα radiation.

5. The co-crystal according to claim 3, wherein the X-ray powder diffraction pattern comprises at least one characteristic peak at 2theta values of 10.9°±0.2°, 12.6°±0.2° and 22.8°±0.2° using Cu-Kα radiation.

6. The co-crystal according to claim 1, wherein the X-ray powder diffraction pattern is substantially as depicted in FIG. 1 using Cu-Kα radiation.

7. The co-crystal according to claim 1, which is an anhydrous co-crystal.

8. A pharmaceutical composition, wherein said pharmaceutical composition comprises a therapeutically effective amount of co-crystal according to claim 1, and pharmaceutically acceptable excipients.

9-10. (canceled)
